(19) United States
(12) Patent Application Publication
(10) Pub. No.: US 2008/0268503 A1
(43) Pub. Date: Oct. 30, 2008

(54) Method for Achieving High-Level Expression of Recombinant Human Interleukin-2 Upon Destabilization of the Rna Secondary Structure

(75) Inventors: Mitali Samaddar, Andhra Pradesh (IN); Sheeram Nallar Chakravarthy, Andhra Pradesh (IN); Srilalitha Movva, Andhra Pradesh (IN); Jayalakshmi Gosala, Andhra Pradesh (IN); Sreenivasu Karra, Andhra Pradesh (IN); Uma Devi Komath, Andhra Pradesh (IN); Jayaram Chigurupati, Andhra Pradesh (IN)

Correspondence Address: VENABLE LLP, P.O. BOX 34385, WASHINGTON, DC, 20043-9998, US

(73) Assignee: ZENOTECH LABORATORIES LIMITED, ANDHRA PRADESH (IN)

(21) Appl. No.: 11/886,416

(22) PCT Filed: Mar. 17, 2006
(86) PCT No.: PCT/IN06/00098

§ 371 Date: Sep. 17, 2007

(30) Foreign Application Priority Data
Mar. 17, 2005 (IN) 273/CHE/2005

(51) Int. Cl. C12P 21/04

(52) U.S. Cl. 435/69.52

(57) ABSTRACT

The present invention provides a method for achieving high-level expression of the therapeutically important lymphokine (human IL-2). The method comprises of identifying the secondary structure in the 5′ region of human IL-2 mRNA, modifying the 5′ region of the human IL-2 DNA sequence to produce a new DNA sequence wherein the mRNA transcribed from the modified human IL-2 DNA sequence has the predicted 5′ secondary structure destabilized with increased free energy compared to that of the secondary structure of the mRNA transcribed from the native DNA sequence without altering the sequence of the encoded amino acids; and using this modified DNA sequence of human IL-2 for high level recombinant expression in a microbial host for large scale production. This method is also applicable to other expression host like yeasts and mammalian cells.

### FIELD OF THE INVENTION

The present invention provides a method for achieving high-level expression of human Interleukin-2 (IL-2) in heterologous hosts like bacteria, yeasts etc. by obliterating a translational block due to an identified RNA secondary in the 5′ region of the gene sequence. The said method comprises of identifying the secondary structure of the mRNA in the 5′ region of the gene, modifying the sequence to destabilize the secondary structure without altering the encoded amino acid sequence and using the said modified sequence in the recombinant expression system for protein production.

### BACKGROUND OF THE INVENTION

The human Interleukin-2 (IL-2), also called as the T-cell growth factor, is a lymphokine whose activity allows the long-term proliferation of T-cells following interaction with antigen, mitogen or alloantigen (Smith, K. A. Immun. Rev. (1980) 51, 337-357). It is synthesized and secreted by activated T-lymphocytes and has been purified from various sources such as human peripheral blood lymphocytes, tonsilar lymphocytes, spleen lymphocytes, T-cell leukemia and T-cell hybridoma cultures (Gillis et al., J Immunol (1978) 12; 2027-2032). The human IL-2 when purified from native sources is found to have molecular weight in the approx range of 13,000 to 17,000 daltons and isoelectric point in the approximate range of pH 6.0-pH 8.5 (S. Gillis and J. Watson, J Exp Med (1980) 159: 1709-1719), This heterogeneity can be attributed to differences in the extent of glycosylation of the protein. This posttranslational modification does not seem to be necessary for biological activity of the hormone (U.S. Pat. No. 5,614,185). Human IL-2 when expressed in a microbial host is not glycosylated and is produced in a reduced state. When purified and oxidized these microbially produced IL-2s exhibit activity comparable to native human IL-2 (U.S. Pat. No. 5,614,185).

In addition to being a T-cell growth factor, human IL-2 is also known to have other biological activities such as enhancement of thymocyte mitogenesis (Chen et al., Cell Immunol (1977), 22: 211-224; Shaw et al., J Immunol (1977) 22; 211-224), induction of cytotoxic T-cell reactivity (Wagener et al., Nature (1980) 284: 278-280 and U.S. Pat. No. 4,738,927). These activities indicate that human Interleukin-2 can play a significant role in immuno therapy against bacterial or viral infections, and immune deficient disease as it regulates the functions of immune system. Thus, human IL-2 is being pursued as a very important therapeutic drug in treating cancer and other immune related diseases. The drug Proleukin (Aldesleukin) from Chiron comprises of biologically active human IL-2 that is indicated for treatment of adults with metastatic renal cell carcinoma and metastatic melanoma. In addition large number of clinical trials in various phases are ongoing wherein IL-2 is being used as an immuno-stimulator for HIV therapy, vaccines, stem cell therapy, cancer etc.

The ever-increasing acceptance of human IL-2 as a drug for large number of indications creates a necessity to develop very efficient recombinant expression systems that allows production of large quantities of therapeutic grade lymphokine. The human IL-2 gene has been isolated, cloned at a position downstream of a promoter sequence of a vector by using rDNA technology and expressed in microorganism or eukaryotic cells (Taniguchi et. al., Gene, (1980) 10; 11-15, Taniguchi et. al., Nature 1983, 302; 305-310; Devos, Nucleic acids research 1983, 11: 4307-4323, U.S. Pat. No. 4,738,927). Glycosylated protein of IL-2 mutein (substitution of “Asp” at 88 position with “Arg”) has been produced using mammalian cells that may have therapeutic application requiring activation of immune system (U.S. Pat. No. 6,348,192). The bacterial expression system has been the workhorse for expression of therapeutic and commercially important proteins. Thus, various groups have extensively used this system to express human IL-2 in biologically active form. The hIL-2 when isolated from bacterial cells is in the form of an aggregated oligomeric and multimeric form that has to be reduced with reducing agents. This can be explained by the fact that human IL-2 has three cysteine residues at positions 58, 105 and 125 and two of them form the single disulfide bridge with one free cysteine. The presence of one extra cysteine contributes to intermolecular crosslinking or incorrect disulfide bridge formation. Studies carried out using site-directed mutagenesis to mutate one of the cysteine residues showed that the cys125 is not involved in the disulfide bond formation and can be mutated to serine without any loss of activity. The U.S. Pat. Nos. 4,959,314, 4,853,332, and 4,518,584 describes the construction of human Interleukin-2 (hIL-2) muteins, by site directed mutagnesis at cysteine amino acid positions 58, 105, and 125 either by deletion or replacing with neutral amino acid such. as serine. The presence of three cysteines means that the protein may randomly form one of the three intra molecular disulfide bonds, but only one of those being the correct as found in the native molecule. The bio assay activity of the expressed protein showed that cysteine residues at positions 58, and 105 are necessary for biological activity. Thus the human Interleukin-2 mutein having serine instead of cysteine at 125th position, and also lacking the N-terminal alanine residue (N-terminal methionine is removed during the processing) is biologically active.

The levels of expression achieved for human IL-2 when expressed in a microbial host using the wild type gene sequence have been found to be relatively low when compared to proteins that are well expressed. Devos et al., Nucl Acid Res (1983) 11, 4307-4323, has carried out expression of human IL-2 in bacterial host using two different promoter systems. The bacterially expressed human IL-2 was biologically active but the level of expression obtained was only 5% to 10% of the total cellular protein in a best-case scenario. This low level expression of the lymphokine can be responsible for less efficient purification procedures leading to an expensive and cumbersome production process. Thus, it becomes necessary to investigate and identify the cause of such low level expression followed by manipulations to overcome such constraints to elevate the levels of expression of human IL-2.

The microbe Escherichia coli as an expression host provides a process for production of recombinant proteins that requires a simple process and design with enormous economic advantages. However, inspite of the extensive understanding of the genetics and molecular biology of E. coli, not every gene can be expressed efficiently in this organism. This may be due to unique and subtle structural features of the gene sequence. the stability and translatability of mRNA, the ease of protein folding, protein degradation by host cell proteases, major differences in codon usage between the foreign gene and native E. coli and the potential toxicity of the protein to the host (Makrides C. S. (1996) Microbilogical Reviews, September 512-538). One of the most important factor affecting translatability of the mRNA is the ability of the gene sequence and the 5 regulatory sequence to form stable RNA secondary structures. The process of translation is initiated by binding of ribosomes to the Shine-Dalgarno sequence on the mRNA and this is followed by synthesis of polypeptide starting from the start codon ‘AUG’. It has been shown that the secondary structure of the mRNA in the region of the Shine-Dalgarno sequence and the 5′ region of the gene sequence plays a critical role in determining the efficiency of translation. The formation of stable stem loop structures in the 5′ region of the message could obstruct the assembly and impede the movement of the ribosome complex thus inhibiting translation. Removal or destabilization of such attenuating sites could significantly improve translation efficiency resulting in high-level expression of the recombinant protein.

This embodiment comprises of a method to achieve high-level expression of human IL-2 in bacteria by significantly improving the translation efficiency of the mRNA. The method involves identifying stable secondary structure in the 5′ region of the gene downstream of the Shine-Dalgarno sequence, specifically modifying the DNA sequence without changing the encoded amino acid sequence so as to destabilize the identified secondary structure. This modified sequence of human IL-2 (des Ala and Cys125Ser) when expressed in bacterial host results in high-level expression of the lymphokine accounting for about 40% to 50% of the total cellular protein which is about 4 to 5 fold higher levels than what has been reported (Devos et al., Nucl Acid Res (1983) 11, 4307-4323). Thus this invention has enormous implications in the use of human IL-2 as a therapeutic drug for many indications as it provides a very efficient recombinant expression system for a simple and economic production process.

### SUMMARY OF THE INVENTION

The present invention provides a method for achieving high-level expression of the therapeutically important lymphokine (human IL-2). The method comprises of identifying the secondary structure in the 5′ region of human IL-2 mRNA, modifying the 5′ region of the human IL-2 DNA sequence to produce a new DNA sequence wherein the mRNA transcribed from the modified human IL-2 DNA sequence has the predicted 5′ secondary structure destabilized with increased free energy compared to that of the secondary structure of the mRNA transcribed from the native DNA sequence without altering the sequence of the encoded amino acids; and using this modified DNA sequence of human IL-2 for high level recombinant expression in a microbial host for large scale production. This method is also applicable to other expression host like yeasts and mammalian cells.

The native DNA sequence may be modified at the 5′ end of the coding sequence about 90 to about 60 nucleotides from the initiation codon. A list of DNA sequences and their corresponding free energy is generated ensuring that the encoded amino acid sequence is not altered. From the pool of such altered DNA sequences, the sequences that have higher free energy compared to the native sequence are selected. Once the synthetic DNA sequences containing the desired optimized sequence is constructed, the gene is inserted into an appropriate expression vector. by standard techniques (Sambrook et al. (1989) Molecular Cloning. A Laboratory Manual, Cold Spring Harbor Laboratory Press). The engineered expression constructs are used to transform a bacterial host like the B-derived strain of E. coli. which is used for high-level expression of human IL-2.

Accordingly, the present invention provides a method for achieving high-level recombinant expression of therapeutically important lymphokine human IL-2 such that the bacterially expressed human IL-2 has a modified DNA sequence that obliterates the translation block observed with the native sequence, said method comprising
a) identifying the secondary structure in the 5′ region of the mRNA transcribed from a mature human IL-2 DNA sequence that impedes the assembly and or movement of the ribosome complex,
b) modifying the said DNA sequence of the 5′ region of human IL-2 DNA to produce a new DNA sequences wherein the said mRNA transcribed from the modified human IL-2 DNA sequence has the predicted 5′ secondary structure destabilized with increased free energy compared to that of the secondary structure of the mRNA transcribed from the native DNA sequence without altering the sequence of the encoded amino acids
c) using the said modified DNA sequence of human IL-2 for cloning to obtain the human IL-2 expression construct and transformation of microbial host with the said human IL-2 expression construct for high level recombinant expression in a microbial host for large scale production

The said modified DNA sequence is cloned under the transcriptional control of an inducible promoter in an expression vector to generate the human IL-2 expression construct.

The said microbial host is preferably the B derived strain of E. coli.

### DESCRIPTION OF THE ACCOMPANYING DRAWINGS

The invention will now be described with reference to the accompanying drawings

FIG. 1a shows the wild type DNA and the encoded amino acid sequence of mature human IL-2 (des Ala and Cys125Ser)

FIG. 1b is the wild type DNA sequence for 5′ region of the IL-2 mRNA

FIG. 1c, d and e are the modified DNA sequences for the 5′ region of the IL-2 mRNA

FIG. 2 is the RNAfold-predicted RNA secondary structures with free-energy values for the first 30 nucleotides of mRNA encoding human IL-2 preceded by 30 nucleotides that contains the ribosome binding site (RBS). The nucleotides of the IL-2 portion are shown in bold and the nucleotides that have been changed, but coding for the same amino acid as the wild-type sequence. are underlined. (a) wild-type mRNA sequence, (b) codons 2, 3, 4, 5 & 7 are changed. (c) codons 2, 3, 4, 5, 7 & 10 are changed and (d) codons 2, 3, 5, 7 & 10 are changed.

FIG. 3 is the map of the bacterial expression vector.

FIG. 4 is SDS-PAGE analysis of recombinant human IL-2 in E. coli. (Bacterially expressed human IL-2 is marked by arrow) The proteins were visualized upon staining with commassie blue dye.

FIG. 4a is the expression profiles with wild-type hIL-2 DNA sequence. Lane 1: uninduced control, Lane 2: 100 M IPTG-induced, Lane 3: 250 M IPTG-induced, Lane 4: 500 M IPTG-induced, Lane 5: 1 mM IPTG-induced, Lane 6: Mol Wt Marker

FIG. 4b. is the expression profiles with modified hIL-2 DNA sequence. Lane 1: uninduced control, Lane 2: 100 M IPTG-induced, Lane 3: 250 M IPTG-induced, Lane 4: 500 M IPTG-induced, Lane 5: 1 mM IPTG-induced, Lane 6: Mol Wt Marker (marked by arrow is the E. coli-expressed human IL-2)

FIG. 5 is the western blot analysis using anti-human IL-2 monoclonal antibody

Lane 1 and 2: wild type human IL-2 sequence induced with 100 M IPTG and 250 M IPTG respectively, lanes 3 and 4: modified human IL-2 sequence induced with 100 M IPTG and 250 M IPTG respectively (marked by arrow is the immunoreactive E. coli-expressed human IL-2)

### DETAILED DESCRIPTION OF THE INVENTION

The present invention provides a method for achieving high-level expression of the therapeutically important lymphokine (human IL-2). The first step involves scanning the 5′ region of the mRNA in conjunction with about 20-30 bases upstream of the start codon comprising of the Shine Dalgarno sequence (Ribosome Binding Site) and sequences after the transcriptional start site. The method comprises of identifying the secondary structure in the 5′ region of human IL-2 mRNA. Having identified the important bases involved in stabilizing the secondary structure, appropriate base changes are introduced that destabilizes the secondary structure without changing the encoded amino acids. The human IL-2 mRNA with destabilized secondary structure is speculated to have improved translational efficiency resulting in significantly higher levels of expression.

The experimental procedures comprise of isolating the coding sequence of human IL-2 from human T-cell derived Jurkat cell line upon stimulation with concavalin A or Phorbol myristate acetate (PMA) or any other source like peripheral lymphocytes, followed by cloning into an expression vector. Using appropriately modified oligonucleotides the modified sequences were used to replace the wild type sequence. Restriction mapping and DNA sequencing were used to confirm the identity of all the cloned sequences for human IL-2. These plasmid constructs were used to transform the B derived strain of E. coli. The transformed cells were grown in suitable media like TB or LB or a completely defined medium and expression of human IL-2 was induced upon addition of inducers like lactose and IPTG. The induced lymphokine was detected by SDS-PAGE analysis of the total cell protein. The immunological identity of the protein was confirmed by western blotting using a commercially available monoclonal antibody against human IL-2.

The expression levels of human IL-2 using the constructs with modified sequences were significantly higher than the level that was obtained for the wild type sequence. Experimental data showed almost 5 to 6 fold increase in expression levels when compared to the wild type sequence. This invention provides a highly improved expression system for human IL-2 that can enormously improve the production process for this therapeutic molecule.

### EXAMPLE 1

### Prediction of mRNA Secondary Structure

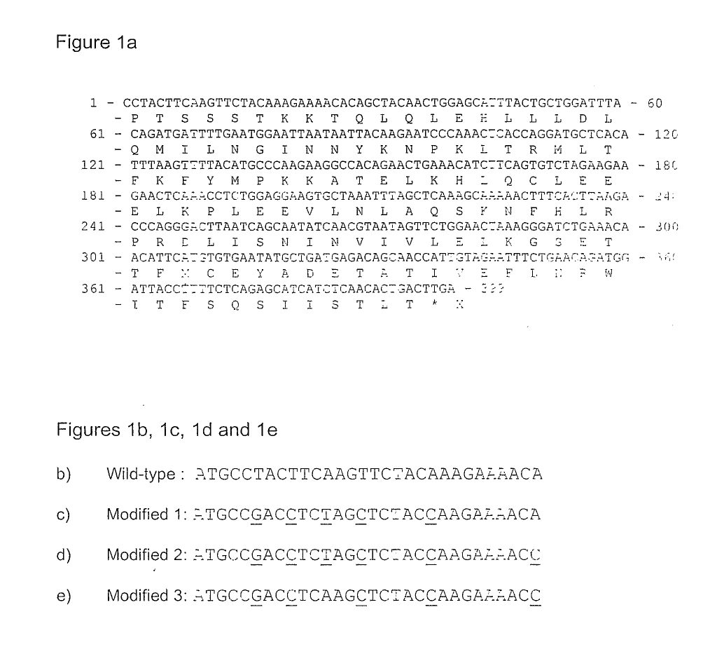

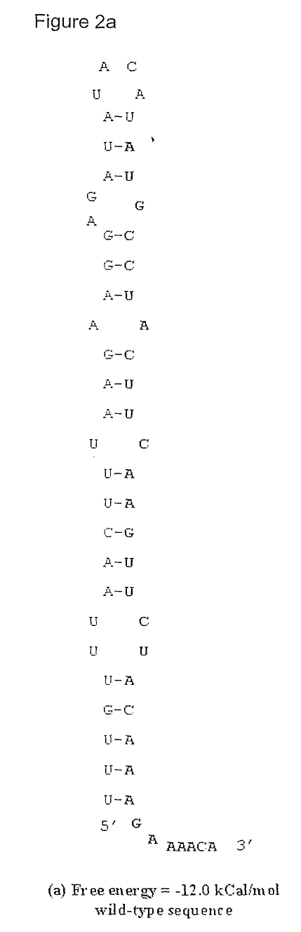

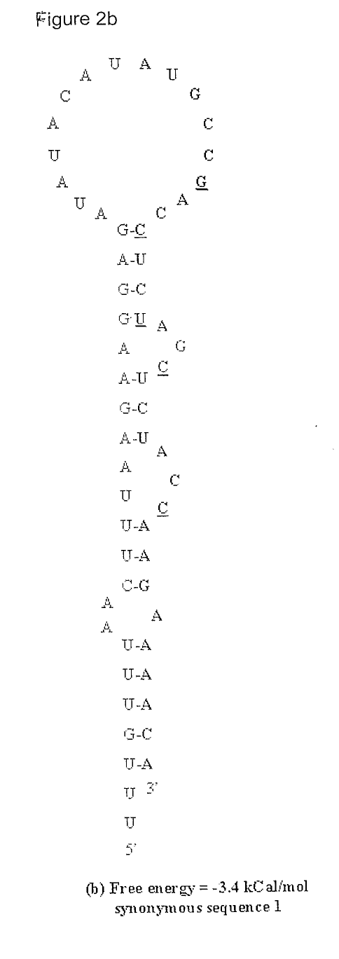

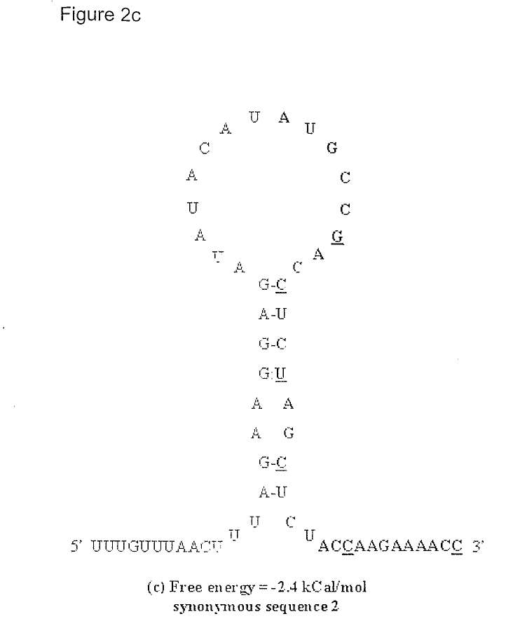

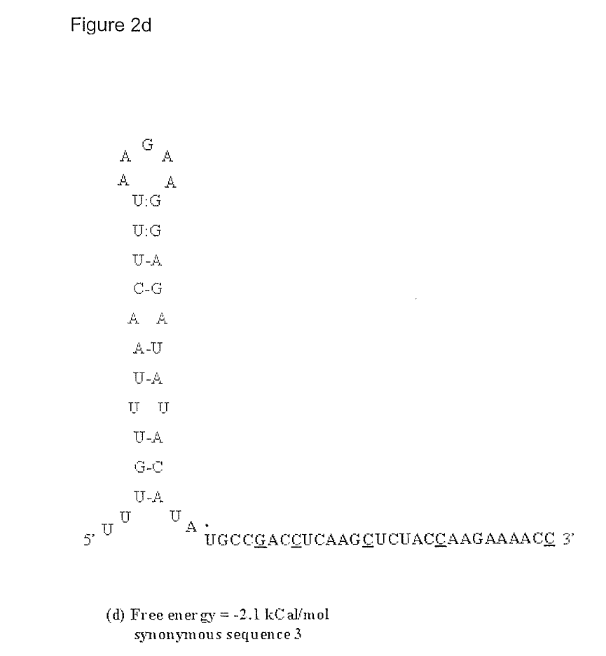

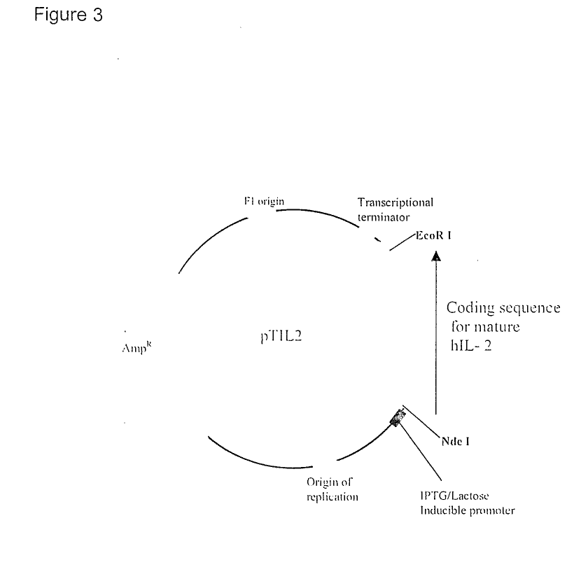

This example describes the procedure followed to identify the secondary structure in the 5′ region of the human IL-2 mRNA that is capable of obstructing translation and is responsible for low-level expression of the protein. A region of about 100-150 was used for RNA secondary structure predictions and free energy calculations using the software called RNAfold developed by Hofacker I L et al. The method involves RNA secondary structure prediction through energy minimization (Hofacker, I L et al. (1994) Monatshefte f. Chemie. 125:167-188; Zuker, M and Stiegler, P (1981) Nucl Acid Res, 9: 133-148; McCaskill J S (1990) Biopolymers, 29: 1105-1119). Based on the analysis, a 60-base window was defined to have a propensity to form a stable stem-loop structure capable of impeding the ribosome and thus obstructing translation that is coupled to transcription. Using the degeneracy of the genetic codons, various base changes were incorporated that increased the free energy in the region without altering the encoded amino acids. All the codon(s) for an amino acid were used in various combinations and the free energy of the structures comprised an array from which the described sequences were selected that had higher free energy compared to the wild-type sequence, thus destabilizing the secondary structure. The nucleotide and amino acid sequence of the wild-type hIL-2 gene is given in FIG. 1. The RNA secondary structures and the free energy of wild type and modified sequence(s) are given in FIG. 2.

### EXAMPLE 2

### Cloning of hIL-2 Gene

In the present embodiment, the mature coding portion of the human IL-2 gene is isolated from the mammalian cells that produce IL-2 such as the Jurkat cells derived from leukemic T lymphocytes, or peripheral lymphocytes. Suitable stimulants include mitogens, neuraminidase, galactose oxide, zinc derivatives such as zinc chloride. After 3-12 hours after inductions the cells are lysed and total RNA is extracted from the cells and converted into cDNAs. An aliquot of the synthesized cDNA is used as a template for amplifying the desired DNA fragment of human IL-2 coding sequence using appropriately designed specific oligonucleotide primers. The human IL-2 amplicon is cloned into the expression vector suitably placed with respect to the transcription and translation signals. An IPTG or lactose inducible promoter drives the transcription of the human IL-2 coding sequence. Using the wild type construct as the parent sequence, the required base changes described for the modified sequences were incorporated using appropriately designed oligonucleotide primers. Microbial host strains like the B-derived strain of E. coli harboring the plasmid constructs produces mature human IL-2 when induced with lactose or IPTG.

### EXAMPLE 4

### Expression of Human IL-2

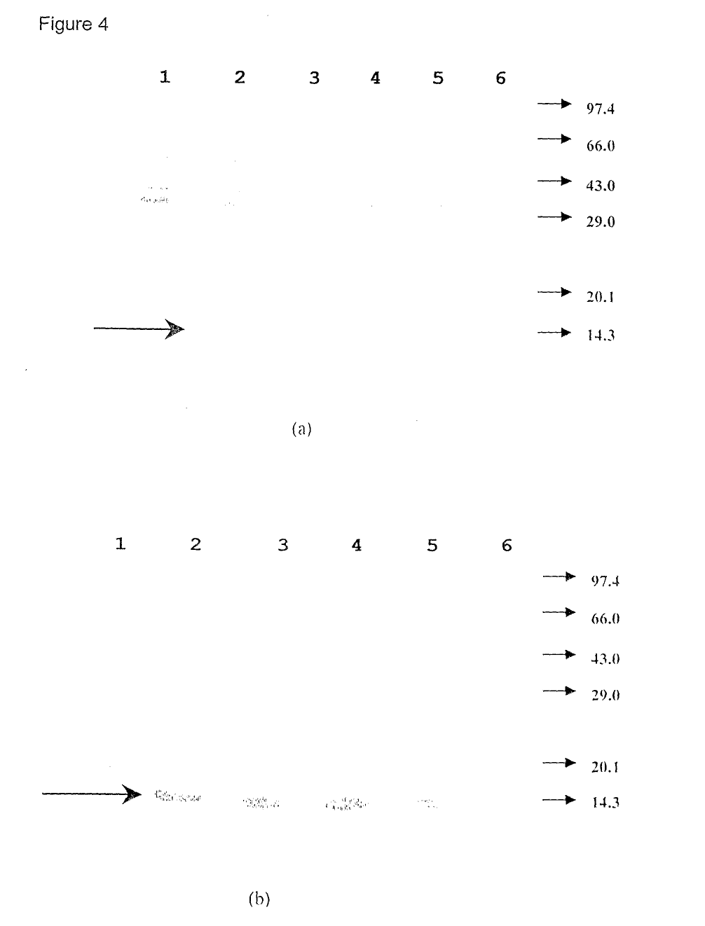

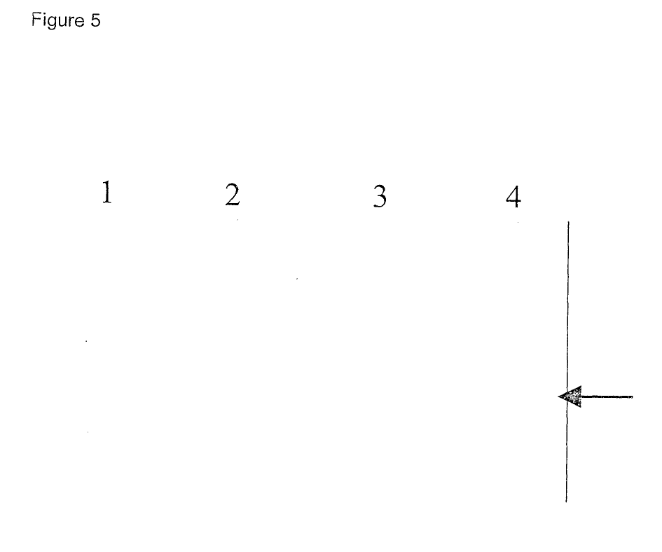

This example relates to the dramatic improvement in expression of human IL-2 using the constructs with modified DNA sequences. E. coli expression hosts were transformed with the recombinant plasmid constructs using standard procedures known in the art. A well-isolated colony was picked from the plate and grown overnight at 37° C. in LB or TB or completely defined media. Fresh media were inoculated with the overnight cultures and grown at 37° C. till O.D.₆₀₀ reached ˜1.0. The cultures were induced by adding IPTG (100 M to 1 mM final concentration) or lactose (1 mM to 100 mM) and grown for 4 hours at 37° C. At the end of the induction period cells were harvested and an aliquot of the cell lysate was analyzed by SDS-PAGE. The protein profile of various samples was visualized by staining with the commassie blue dye. As is seen in FIG. 4 the expression levels for human IL-2 dramatically increased (5-6 fold) when plasmid constructs containing the modified DNA sequences encoding the same amino acids for human IL-2 were used. This finding further reinforces the role of stable secondary structures in the 5′ region of the gene responsible for low-level expression in a host with coupled transcription and translation machinery. Thus this invention provides a novel method of obtaining high levels of recombinant human IL-2 for therapeutic production of the lymphokine using a cost-effective and a highly efficient process.

### EXAMPLE 5

### Immunological Identity of Recombinant hIL-2

This example relates to immunological identity of the expressed protein using a commercially available monoclonal antibody specific to human IL-2. The Western blot analysis was carried out using standard procedures known in the art. The cell lysates from equal number of induced cells were used for the analysis. The E. coli-expressed human IL-2 using both wild type and modified sequence constructs showed very specific reactivity to the monoclonal antibody, thus confirming the immunological identity of the expressed protein. Moreover there was almost 5 to 6 fold increase in the intensity of the signals for the modified sequence when compared to that of the wild type (Quantity One software). This data further confirmed that human IL-2 when expressed using the modified sequences resulted in high-level expression and almost 5 to 6 fold higher levels to that when wild type sequence is used. Thus, this invention provides a very simple means of achieving high-level expression of recombinant human IL-2 for therapeutic applications.

We claim:

1. A method for achieving high-level recombinant expression of therapeutically important lymphokine human IL-2 such that the bacterially expressed human IL-2 has a modified DNA sequence that obliterates the translation block observed with the native sequence, said method comprising
a) identifying the secondary structure in the 5′ region of the mRNA transcribed from a mature human IL-2 DNA sequence that impedes the assembly and or movement of the ribosome complex,
b) modifying the said DNA sequence of the 5′ region of human IL-2 DNA to produce a new DNA sequences wherein the said mRNA transcribed from the modified human IL-2 DNA sequence has the predicted 5′ secondary structure destabilized with increased free energy compared to that of the secondary structure of the mRNA transcribed from the native DNA sequence without altering the sequence of the encoded amino acids
c) using the said modified DNA sequence of human IL-2 for cloning to obtain the human IL-2 expression construct and transformation of microbial host with the said human IL-2 expression construct for high level recombinant expression in a microbial host for large scale production

2. The method as claimed in claim 1, wherein the said modified DNA sequence is cloned under the transcriptional control of an inducible promoter in an expression vector to generate the human IL-2 expression construct.

3. The method as claimed in claim 1 wherein the said microbial host is preferably the B derived strain of E. coli.

4. A method for achieving high-level recombinant expression of therapeutically important lymphokine human IL-2 substantially as herein described with reference to the accompanying drawings and foregoing examples.
